(19) United States
(12) Patent Application Publication
(10) Pub. No.: US 2026/0263772 A1
(43) Pub. Date: Sep. 10, 2026

(54) CONNECTORS

(71) Applicant: Organox Limited, Oxford (GB)

(72) Inventors: Jeffrey N. Steinmetz, Arvada, CO (US); Jeremy H. Morgan, Missoula, CO (US)

(21) Appl. No.: 19/461,174

(22) Filed: Jan. 27, 2026

Related U.S. Application Data
(60) Provisional application No. 63/750,280, filed on Jan. 27, 2025.

(51) Int. Cl. A61M 39/10

(52) U.S. Cl. CPC A61M 39/1011

(57) ABSTRACT

Various connectors are disclosed for connecting an anatomical conduit, such as a blood vessel of an organ, to a non-biological conduit, such as a conduit of a machine perfusion system. Methods for coupling an anatomical conduit, such as a blood vessel of an organ, to a non-biological conduit, such as a conduit of a machine perfusion system, are disclosed.

### CROSS-REFERENCE TO RELATED APPLICATION

This application claims the benefit of U.S. Provisional Application No. 63/750,280, filed Jan. 27, 2025, the entirety of which is hereby incorporated by reference.

### FIELD OF THE INVENTION

The present disclosure relates to connectors that may be used to connect an anatomical conduit to a non-biological conduit. Such connectors may be used, for example, to connect an artery or a vein of an organ to an external conduit or cannula for machine perfusion.

### BACKGROUND OF THE INVENTION

Organ perfusion technologies are increasingly used in human medicine to preserve biological organs outside the body for diagnostic assessment, transport, and transplantation. Similar techniques may also be used in veterinary contexts. These technologies may employ extracorporeal circulation systems that deliver oxygenated perfusate to an organ while removing venous effluent and other biological fluids. Such systems may operate at hypothermic, subnormothermic, or normothermic temperatures and may be applied a range of organs, including but not limited to kidneys, livers, hearts, lungs, pancreases, and composite tissues. In veterinary applications, similar perfusion approaches are being developed for organs from companion animals, livestock, and research species.

To integrate an explanted organ with a perfusion circuit, one or more anatomical structures—such as arteries, veins, ureters, bile ducts, or other fluid conduits are connected to corresponding elements of the perfusion apparatus. Traditionally, these connections have been made using surgical sutures, graft segments, vascular cannulas, or standardized tubing interfaces. However, such approaches often present a number of challenges. Explanted organs may exhibit significant anatomical variability, including differences in vessel diameters, branching patterns, wall quality, and residual tissue morphology. In many cases, the available vascular stumps may be short, fragile, calcified, or irregular, complicating reliable attachment to standard cannulas or grafts.

Moreover, many existing connection methods are optimized for use in the operating theatre by highly trained surgical personnel. They may involve time-consuming suturing techniques, require careful vessel preparation, or rely on connectors that are not compatible with long-duration perfusion at physiologic pressures and flows. Leakage, kinking, occlusion, vessel trauma, and imperfect sealing may arise, particularly when the connectors are used outside ideal surgical conditions, such as at donor hospitals, retrieval sites, or in laboratory environments.

As perfusion systems evolve toward wider clinical adoption, including deployment in non-specialist settings, there is a need for connection technologies that provide rapid, reliable, and atraumatic engagement with a range of organ types and vessel morphologies. Ideally, such connectors should accommodate substantial anatomical variability, support extended perfusion at physiologic pressures and temperatures, minimize user skill requirements, and avoid introducing mechanical or biological risks to the tissue. A connector that facilitates easy disconnection prior to transplant or subsequent surgical intervention without damaging the native vessels and/or leaving residual materials is also desirable.

Beyond human transplantation, similar challenges exist in veterinary medicine, where organ size, vessel geometry, and species-specific anatomical differences further complicate the use of conventional connection devices. Research and industry groups increasingly require adaptable connector solutions that can be used across diverse species and organ types, whether for preservation, experimental models, regenerative-medicine applications, or development of extracorporeal organ support technologies.

Accordingly, there is a need for improved connection devices and systems that provide secure, adaptable, and user-friendly attachment between biological organs and perfusion or support circuits. The present invention addresses these and other challenges.

The present disclosure pertains generally to connectors for organ preservation during the process of organ transplantation. More particularly, the present disclosure pertains to vascular connectors capable of removably coupling to arteries and veins of organs harvested from organ donors, connecting the arteries and veins to preservation systems, and being removed or being used during transplantation into a transplant patient.

### SUMMARY OF THE INVENTION

The vascular connectors of the present disclosure consist generally of a first connector and a second connector, wherein the first connector and the second connector interface with each other in a sealing manner to provide a fluid tight conduit between an organ blood vessel and a perfusion conduit that supplies a perfusion fluid to an arterial connection and withdraws the perfusion fluid from a venous connection.

A component employed in organ transplantation is a carrel patch that consists of either a portion of a native blood vessel or a synthetic graft which is formed with a flange to allow for connection to a blood vessel in the recipient patient. Certain variants of the connectors of the present disclosure employ a carrel patch-like component.

The first and second connectors of the present disclosure take a variety of configurations. In certain variants of the present disclosure, the first and second connectors are configured to interface with the flange of a carrel patch-like component and retain the flange in a fluid tight manner between the first and second connectors.

According to other variants of the connectors of the present disclosure there is provided a connector having a housing with a de-airing port, a perfusion port, and a cannula configured to receive a blood vessel from the organ being transplanted. An annular member is provided which has suture openings and/or crenellations passing through the annular member and circumferentially surrounding the central opening of the annular member. The suture openings and/or crenellations are configured to receive sutures that are passed through a distal end of the blood vessel from the organ and thereby secure the blood vessel to the connector. A clamp may be employed and placed adjacent to the annular member, to further secure the blood vessel to the cannula.

There is also provided variants of the first and second connectors having different configurations of connector interfaces and different configurations of releasable components that retain the first and second connectors in a fluid tight connection

Finally, there are provided perfusion tubing handling components that allow for supported bends in the perfusion tubing to allow the fusion tubing to bend without kinking. Also provided is an angular housing connector that may, optionally, replace the perfusion tubing handling component.

It is an object of the present disclosure to provide variants of vascular connectors for organ preservation after organ transplant, during transportation of the explanted organ in an organ preservation system, and to facilitate transplantation of the organ and the recipient patient.

According to a first aspect, there is provided a connector for coupling an anatomical conduit to a non-biological conduit, the connector comprising:
a first connector portion comprising a conduit connector for coupling to a non-biological conduit;
a second connector portion; and
one or more ratcheting fasteners configured to clamp the second connector portion to the first connector portion with the portion of the anatomical conduit between the first connector portion and the second connector portion and in sealing engagement with a sealing region of the first connector portion.

Each ratcheting fastener may comprise:
a flexible arm comprising at least one tooth, the flexible arm fixed at a proximal end of the flexible arm to one of the first connector portion and the second connector portion, and
fixed teeth on the other of the first connector portion and the second connector portion.

The ratcheting fastener may be operable to clamp the second connector portion to the first connector portion with a clamping force that depends on a position of engagement of the at least one tooth with the fixed teeth.

Each ratcheting fastener may be operable to disconnect and unclamp the first connector portion from the second connector portion.

The one or more ratcheting fasteners may comprise a plurality of self-locking ratcheting fasteners spaced apart around the sealing region.

The flexible arm may comprise a plurality of teeth.

The portion of the anatomical conduit may be a flange of a carrel patch.

The sealing region may comprise an annular region of the first connector portion.

The annular region may be a generally oval annular region of the first connector.

The conduit connector may be is coaxial with the sealing region of the first connector portion and/or a plane of the sealing region may be substantially normal to the axis of the conduit connector.

The sealing region may comprise one or more ridges or grooves.

The second connector portion may comprise a corresponding sealing region with a complementary shape to the sealing region of the first connector portion. The sealing region of the first connector portion and the second connector portion may be together configured to promote a sealing engagement between the anatomical conduit and the first connector portion.

The connector may further comprise a de-airing port for removing air from the connector when the connector is coupling the anatomical conduit to the non-biological conduit.

The first connector portion may comprise the de-airing port, and the de-airing port may be between the sealing region and the conduit connector.

The connector may be configured to connect a blood vessel of an organ to a machine perfusion system.

The organ may be a human kidney or a human liver.

According to a second aspect, there is provided a connector for coupling an anatomical conduit to a non-biological conduit, the connector comprising:
a connector body comprising a conduit connector for coupling to a non-biological conduit,
a flexible patch comprising a through hole defining an inner perimeter, and
a patch retainer;
wherein the connector is configured to clamp at least the inner perimeter of the flexible patch between the connector body and the patch retainer to form a sealing engagement between the flexible patch and the connector body; and
the connector comprises a fluid channel through the through hole and the patch retainer to the conduit connector.

The flexible patch may be configured to be sutured in sealing engagement with the patch of the anatomical conduit, thereby coupling the anatomical conduit to the conduit connector.

The patch retainer may be is retained in an inlet port of the connector body, clamping at least the inner perimeter of the flexible patch between the connector body and the patch retainer, by an interference fit between the patch retainer and the inlet port.

The patch retainer may be retained in a recess of the connector body, clamping at least the inner perimeter of the flexible patch between the connector body and the patch retainer, by a threaded engagement between the patch retainer and the inlet port.

The connector may further comprise a flexible sealing element configured to form the sealing engagement between the flexible patch and the connector body.

The conduit connector may be coaxial with the through hole of the flexible patch and/or a plane of the flexible patch may be substantially normal to the axis of the conduit connector.

The connector body may comprise a de-airing port, and the de-airing port may be between the flexible patch and the conduit connector.

The connector may be configured to connect a blood vessel of an organ to a machine perfusion system.

The organ may be a human kidney or a human liver.

According to a third aspect, a connector is provided for coupling an anatomical conduit to a non-biological conduit, the connector comprising:
a connector body comprising a conduit connector for coupling to a non-biological conduit;
a spigot for insertion into the anatomical conduit and making a sealing engagement between an interior surface of the anatomical conduit and an outer surface of the spigot;
a retaining arrangement for securing the anatomical conduit in sealing engagement with the spigot.

The retaining arrangement may comprise a shoulder on the spigot so that the spigot is configured as a barb.

The retaining arrangement may comprise a suture in the anatomical conduit wrapped around a portion of the connector body.

The conduit connector may be coaxial with the spigot. The connector may further comprise an insertion dilator for inserting through the conduit connector and the spigot into a lumen of the anatomical conduit to improve the ease of engaging the anatomical conduit with the spigot.

The conduit connector may comprise a quick coupling arrangement or a barb.

According to a fourth aspect, there is provided a method of connecting a blood vessel of an organ to a perfusion system, the method comprising:
positioning a second connector portion of a connector around the blood vessel with a portion of the blood vessel between the second connector portion and a first connector portion of the connector;
clamping the second connector portion to the first connector portion with the portion of the blood vessel between the first connector portion and the second connector portion and in sealing engagement with a sealing region of the first connector portion;
connecting a conduit of the perfusion system to a conduit connector of the connector.

The organ may be a kidney or a liver. The connector may be according to the first aspect, including any optional features thereof.

According to a fifth aspect, there is provided a method of connecting a blood vessel of an organ to a perfusion system, the method comprising:
suturing a flexible patch of a connector in sealing engagement with a patch of the blood vessel;
connecting a conduit of the perfusion system to a conduit connector of the connector;
wherein the connector comprises:
   a connector body comprising the conduit connector,
   the flexible patch, comprising a through hole defining an inner perimeter, and
   a patch retainer;
   wherein the connector clamps at least the inner perimeter of the flexible patch between the connector body and the patch retainer to form a sealing engagement between the flexible patch and the connector body; and
   the connector comprises a fluid channel through the through hole and the patch retainer to the conduit connector.

The organ may be a kidney or a liver. The connector maybe according to the second aspect, including any optional features thereof.

According to a sixth aspect, there is provided a method of connecting a blood vessel of an organ to a perfusion system, the method comprising:
engaging the blood vessel with a spigot of a connector and making a sealing engagement between an interior surface of the blood vessel and an external surface of the spigot;
securing the blood vessel in sealing engagement with the spigot using a retaining arrangement;
connecting a fluid conduit of the perfusion system to a conduit connector of the connector.

The organ may be a kidney or a liver. The connector may be according to the third aspect, including any optional features thereof.

The features of each aspect, example or variant may be combined with those of any other aspect, example or variant, including optional features thereof.

### BRIEF DESCRIPTION OF THE DRAWINGS

FIG. 1 is a perspective exploded view of a first variant of the connector of the present disclosure.

FIG. 2 is a perspective view of the first variant of the connector in its assembled state.

FIG. 3 is a perspective exploded view of a second variant of the connector of the present disclosure.

FIG. 4 is a perspective exploded view of a third variant of the connector of the present disclosure.

FIG. 5 is a partial cross-sectional view taken along line 5-5 of FIG. 3.

FIG. 6 is an exploded perspective view of a fourth variant of the connector of the present disclosure.

FIG. 7 is a perspective view of the fourth variant of the connector in its assembled state.

FIG. 8 is a partial cross-sectional view of the fourth variant of the connector in its assembled state.

FIG. 9 is a cross-sectional diagram of a fifth variant of the connector of the present disclosure.

FIG. 10 is a partial cross-sectional diagram of a sixth variant of the connector of the present disclosure.

FIG. 11 is a partially exploded perspective view of a seventh variant of the connector of the present disclosure.

FIG. 12 is a partial cross-sectional view taken along line 12-12 in FIG. 11.

FIG. 13 is a partially excluded view of an eighth variant of the connector of the present disclosure.

FIG. 14 is a partially exploded perspective view of a ninth variant of the connector of the present disclosure.

FIG. 15 is a perspective view of the ninth variant of the connector of the present disclosure in its assembled state.

FIG. 16 is an illustration depicting deformation of a first and/or second connector in accordance with the present disclosure.

FIG. 17 is a perspective view of a tenth variant of the connector in accordance with the present disclosure.

FIGS. 18A and 18B are perspective and end elevational views, respectively, of an annular member configured for suture retention of a blood vessel on a cannula in accordance with the tenth variant depicted in FIG. 17.

FIG. 19 is a perspective view of an eleventh variant of the connector in accordance with the present disclosure.

FIGS. 20A and 20B are perspective and an elevation of the use, respectively, of an annular member configured for suture retention of a blood vessel on a cannula in accordance with the eleventh variant depicted in FIG. 19.

FIG. 21 is a perspective view of a twelfth variant of a connector in accordance with the present disclosure.

FIG. 22A is a photographic representation of the twelfth variant of the connector in accordance with the present disclosure.

FIG. 22B is a photographic representation of the twelfth variant of the connector in its fully assembled state.

FIGS. 23A-23E are sequential photographic illustrations depicting the process of joining a blood vessel to the twelfth variant of the connector.

FIG. 24 is a partial cross-sectional side elevational view of a thirteenth variant of the connector in accordance with the present disclosure

FIG. 25 is a side elevational cross-sectional view of the thirteenth variant of the connector depicted in FIG. 24.

FIG. 26 is a partial cross-sectional side elevational view of a fourteenth variant of the connector in accordance with the present disclosure

FIG. 27 is a side elevational cross-sectional view of the fourteenth variant of the connector depicted in FIG. 26.

FIG. 28 is a partial cross-sectional side elevational view of a fifteenth variant of the 10 connector in accordance with the present disclosure

FIG. 29 is a side elevational cross-sectional view of the fifteenth variant of the connector depicted in FIG. 28.

FIG. 30 is a partial side elevational cross-sectional view of a stylet tip in accordance with the fifteenth variant of the connector depicted in FIG. 29.

FIG. 31 is a diagrammatic illustration showing a sixteenth variant of the connector in accordance with the present disclosure.

FIGS. 32 to 35 are photographic depictions of a seventeenth variant of the connector of the present disclosure.

FIG. 36 is a perspective view showing a tube support engaged with a connector in accordance with the present disclosure.

FIG. 37 is a side elevational cross-sectional view of an eighteenth variant of the connector of the present disclosure.

FIG. 38 as a side elevational view of a nineteenth variant of the connector of the present disclosure.

FIG. 39 as a side elevational view of a twentieth variant of the connector of the present disclosure.

FIG. 40A is a perspective view of the first connector portion of the twentieth variant of the connector.

FIG. 40B is a perspective view of a second connector portion of the twentieth variant of the connector.

FIG. 41 as a side elevational view of a twenty-first variant of the connector of the present disclosure.

FIG. 42A is a perspective view of the first connector portion of the twenty-first variant of the connector.

FIG. 42B is a perspective view of a second connector portion of the twenty-first variant of the connector.

FIG. 43 as a side elevational view of a twenty-second variant of the connector of the present disclosure.

FIG. 44A is a perspective view of the first connector portion of the twenty-second variant of the connector.

FIG. 44B is a perspective view of a second connector portion of the twenty-second variant of the connector.

FIG. 44C is a perspective view of a third connector component of the twenty-second variant of the connector in accordance with the present disclosure.

### DETAILED DESCRIPTION

The accompanying Figures depict multiple variants of arterial and venous connectors that are capable of being removably connected to a blood vessel of a donor organ, connected to perfusion conduits for inflow and outflow of a perfusion fluid into the donor organ during transportation in an organ transplant system, and which allow for optional use of the connector or connector components during transplant surgery into a recipient patient.

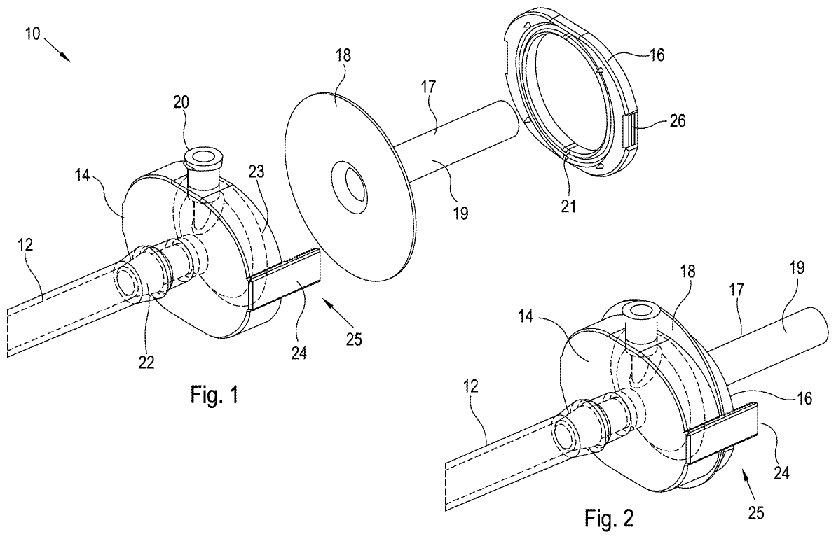

FIGS. 1 and 2 show a connector 10 in accordance with a first variant of the present disclosure. The connector comprises a first connector portion 14 and a second connector portion 16. The connector 10 is for connection to a carrel patch 19 or similar anatomical conduit having a flange 18 connected to a vessel (e.g. a blood vessel). The first connector portion 14 and the second connector portion 16 are configured to fasten together on either side of the 18 flange of the carrel patch 19.

The second connector portion 16 comprises a hole through which the vessel of the carrel patch 19 is received, so that the second connector portion surrounds the vessel. The second connector portion 16 comprises, on a face that is configured to engage with the flange 18, a sealing region 21 surrounding the hole accommodating the vessel (e.g. a groove or ridge, or a flat portion of the second connector portion). The sealing region 21 is configured to make contact with the flange 18 and urge it into sealing engagement with the first connector portion 14. The sealing region 21 may comprise one or more concentric ridges or grooves. Although a circular shape is depicted in FIG. 1 for the ridge forming the sealing region 21, any suitable shape may be used (e.g. elliptical). In the embodiment of FIG. 1 a ridge is shown on the second connector portion 16 but in other embodiments the a sealing feature may comprise a ridge on the first connector portion 14.

The first connector portion 14 is configured to sealingly engage with a surface of the flange 18 that is opposite the vessel of the carrel patch 19. The first connector portion 14 comprises a sealing region 23 for sealing engagement with the flange 18. The sealing region 23 may correspond in shape (e.g. have a complementary shape) with the sealing region 21 of the second connector portion 16.

The first connector portion 14 further comprises a conduit connector 22 in the form of barb 22 for connection to conduit 12 and a de-airing port 20 for removal of air from the connector 10. The de-airing port 20 may include a syringe lock such as a Luer lock.

The conduit 12 may be a perfusion conduit and may comprise flexible tubing that is suitable for machine perfusion of an organ (such as a kidney or a liver).

The first connector portion 14 comprises a tapering (e.g. frusto-conical) region that tapers from the sealing region 23 to an internal lumen of the barb 22. The de-airing port 20 is in fluid communication with the tapering region of the first portion 14 (i.e. between the conduit connector 22 and the sealing region 23.

The first connector portion 14 and the second connector portion 16 of FIGS. 1 and 2 are generally oval shaped (and more specifically stadium shaped or obround), with the de-airing port 20 positioned on a long edge of the oval shape (and specifically on a straight edge of the obround shape). Other shapes are contemplated (e.g. circular or elliptical)—the generally oval shape conveniently accommodates a substantially circular flange 18 and provides some space at the edges of the oval shape (on the tips of the circular portions of the obround) for one or more fastener that maintains the first connector portion 14 and the second connector portion 16 in sealing engagement on the flange 18 of the carrel patch 19.

The one or more fastener in the example of FIGS. 1 and 2 comprises a ratchet fastener 25 on each of opposite sides of the connector 10. Each fastener 25 comprises fixed teeth 26 on an external surface of the second connector 16 and a pawl 24 attached to the first connector portion 14. The pawl 24 comprises a flexible arm carrying teeth, for engagement with the fixed teeth 26. The second connector portion 16 comprises fixed teeth 26 on opposite sides of the exterior of the second connector portion. The first connector portion 14 comprises a pawl 24 at positions on the first connector portion 14 that correspond with the positions of the fixed teeth 26 on the second connector portion 16. As the first connector portion 14 and the second connector portion 16 are brought into engagement, the flexible arm of each pawl 24 urges the teeth of the pawl 24 to engage with the fixed teeth 26. The teeth are shaped so that the pawl 24 can ratchet over the fixed teeth as the first connector portion 14 and the second connector portion 16 are clamped about the flange 18 and to lock and prevent separation of the first connector portion 14 and the second connector portion 16. The first connector portion 14 and the second connector portion 16 can be disengaged by opening the flexible arms outward from the fixed teeth 26 (e.g. by hand, without a tool being necessary).

The fasteners 25 may conveniently be operated (i.e. engaged and/or disengaged) with one hand, and can be used to apply an appropriate sealing engagement force on the flange 18 without resulting in damage to the flange 18.

Each pawl 24 of the one or more fasteners 25 be located on the first connector portion 14 (comprising the barb 22 and de-airing port 20) but the converse arrangement may also be used (in which one or more pawl 24 is carried on the second connector portion 16).

The connector 10 may be used to connect any carrel patch 19 to any conduit 12. Certain embodiments may be particularly suitable for use in machine perfusion of organs including kidneys, but a connector like that of FIGS. 1 and 2 may be used in connecting to any blood vessel.

In embodiments for kidney perfusion, an internal diameter of the sealing region may be less than 7 mm, 6 mm or 5 mm.

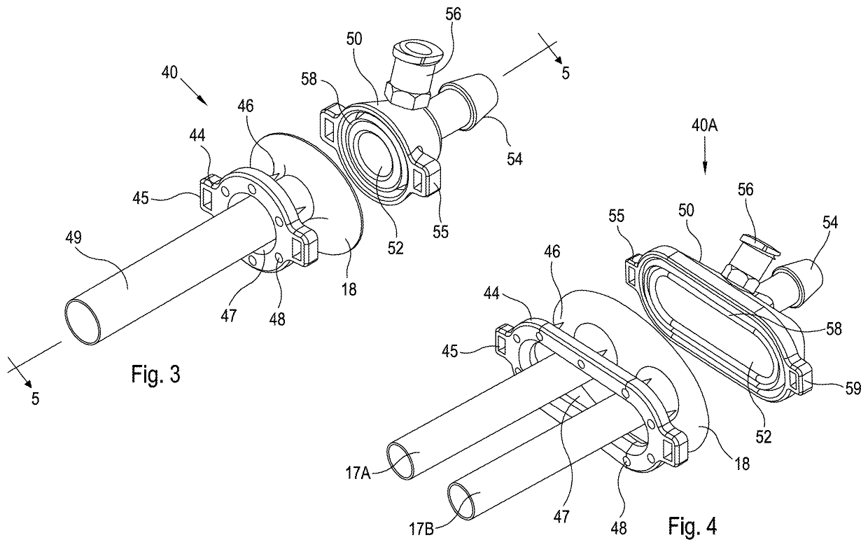

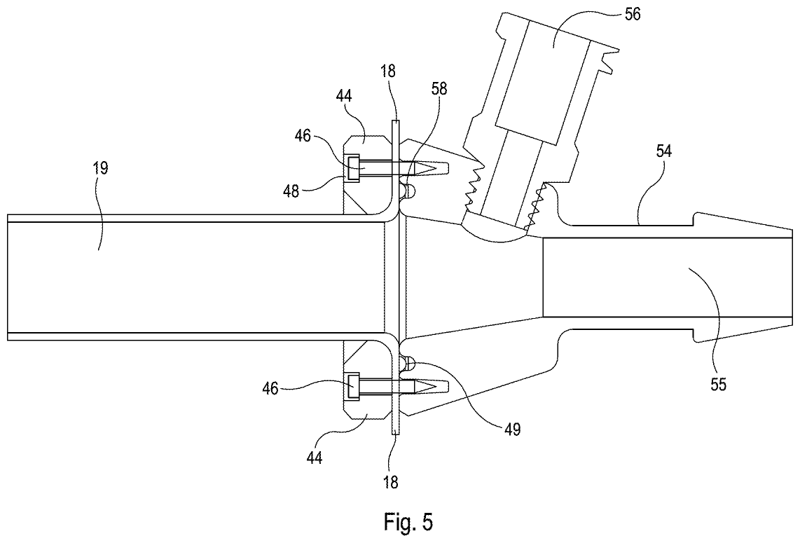

FIGS. 3 and 5 show a second variant of a connector 40 and FIG. 4 shows a third variant 40A which is similar to the connector 40 (and which is labelled with like reference numerals to the second variant). The connector 40, 40A comprises a first connector portion 50 and a second connector portion 44. Similar to the first variant of FIGS. 1 and 2, the connector 40, 40A is configured to sealingly engage with a flange 18 of a carrel patch 19, with the flange 18 disposed between sealing regions of the first connector portion 50 and the second connector portion 44.

The second connector portion 44 comprises a hole 47 through which one or more vessel 17, 17A, 17B of the carrel patch 19 is received, so that the second connector portion 44 surrounds the one or more vessel 17, 17A, 17B on one side of the flange 18. The second connector portion 44 comprises, on a face that is configured to engage with the flange 18, a sealing region feature (comprising a ridge 49) surrounding the hole 47 that receives the vessel. The sealing region of the second connector portion 44 is configured urge the flange 18 into sealing engagement with the first connector portion 50. The first connector portion 50 comprises a sealing region (in the form of a groove 58) corresponding in shape with the sealing region of the second connector portion 44. As illustrated in the sectional view of FIG. 5, the ridge 49 tends to deform the flange 18 into sealing engagement with the groove 58 when the connector 40 is assembled around the carrel patch 19.

As described with reference to the first variant, the sealing region 49, 58 depicted in the second variant and third variant is exemplary and variations are possible.

The first connector portion 50 further comprises a conduit connector 54 (comprising a barb) for connection to conduit (not shown) and a de-airing port 56 for removal of air from the connector 40. The first portion 50 comprises a tapering (e.g. frusto-conical) region that tapers from the sealing region (in contact with the flange 18) to the internal lumen of the conduit connector 54. The de-airing port 56 is in fluid communication with the tapering region of the first connector portion 50.

In the embodiment of FIG. 3, the connector 40 has a generally circular shape, conforming to the shape of a generally circular flange 18 associated with a single vessel. In the embodiment of FIG. 4, the connector 40A has an oval (e.g. obround) shape for accommodating a generally oval (e.g. elliptical) flange 18 associated with two vessels 17A, 17B (as depicted in FIG. 4). The two vessels 17A, 17B are both connected to the barb 54 via the tapering region.

In the second variant and third variant of FIGS. 3 to 5 the flange 18 is secured in position on the second connector portion 44 using barbed members 46. There are a plurality of barbed members 46 that are circumferentially spaced apart, radially outward of the sealing portion of the first and second connector portion 50, 44. A through hole for accommodating each barbed member 46 is provided in the second connector portion 44, and a recess is provided in the first connector portion 50 for receiving a portion of each barbed member 46. The recess in the first connector portion 50 is configured to accommodate each barbed member 46 when the connector 40, 40A is assembled. Each barbed member 46 comprises a head which cannot pass through the through hole of the second connector portion 44.

The recess in the example of FIG. 3 is circular, and there are 6 barbed members 46, spaced equally around the sealing region. Variations on this approach are possible: for example a separate recess may be provided for each barbed member 46 in the first connector portion 50, and more or fewer barbed members may be provided.

The recess in the example of FIG. 4 is obround, and there are 10 barbed members 46, spaced equally around the sealing feature. Again, variations on this approach are possible as described with reference to FIG. 3.

The first and second connector portions 50, 44 further each comprise a pair of lugs that are aligned and adjacent when the connector 50 is assembled. Each lug comprises a through hole that is configured to receive a locking or ratcheting fastener (e.g. a cable tie or similar), thereby securing the first and second connector portions 50, 44 together in sealing engagement with the carrel patch 19.

In FIGS. 3 to 5 the barbed members 46 are shown as separable from the second connector portion 44. The barbed members 46 may preferably be integrally formed with the second connector portion 44. The position of the barbed members 46 and the one or more recess for receiving them may be reversed, so that the barbed members 46 are carried on the first connector portion 50 and inserted into one or more receiving holes or recesses on the second connector portion 44.

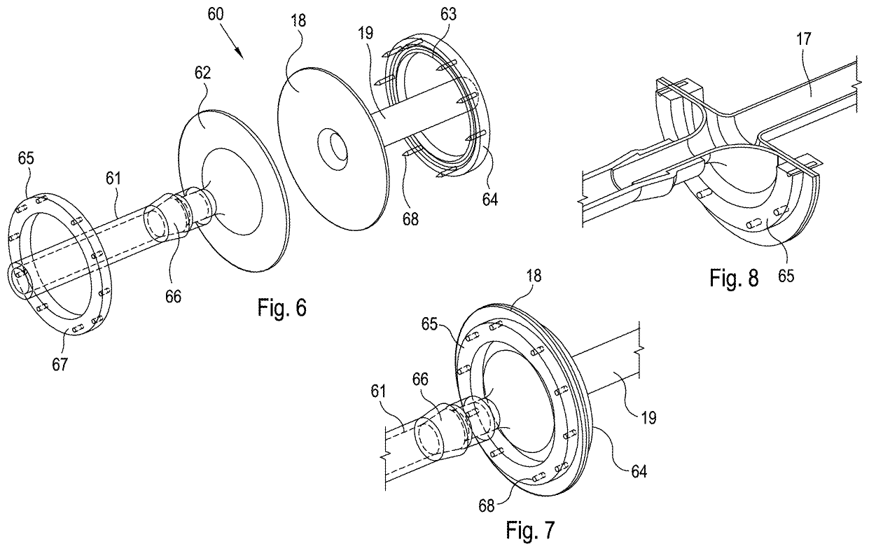

FIGS. 6 to 8 show a connector 60 according to a fourth variant. An exploded perspective view is shown in FIG. 6. The connector 60 is configured to provide a secure and fluid-tight connection between a carrel patch 19 and conduit 61 (which may be used for perfusion). The connector 60 is generally circular in shape.

The connector 60 comprises a first connector portion 62, a second connector portion 64, and a third connector portion 65. The first connector portion 62 includes a barb 66 arranged to engage the perfusion conduit 61 and flares outward from the barb 66 to a flat annular sealing region that is configured to provide a sealing engagement with a surface of the flange 18 opposite to the vessel 17 of the carrel patch 19.

In use, the carrel patch 19 comprising the flange 18 is positioned between the first connector portion 62 and the second connector portion 64. The second connector portion 64 is configured to engage a surface of the flange 18 on the vessel side of the flange 18 and to urge the flange 18 into sealing contact with the sealing region of the first connector portion 62. The second connector portion 64 comprises a sealing region 63 in the form of a circular ridge. The second connector portion 64 comprises barbed members 68 radially outward from the sealing region 63 and spaced circumferentially around the second connector portion 64. There are 10 barbed members 68, spaced equally on a circle, but other numbers of barbed members 68 can be used. The barbed members 68 are integrally formed with the second connector portion 64.

The third connector portion 65 includes recesses or through holes 67 which accommodate the barbed members 68. Each of the barbed members 68 is configured to pass through the flange 18 and engage a respective recess 67 in the third connector portion 65 in an interference fit. As a result, the flange 18 and the flange seal may be securely clamped between the first connector portion 62 and the second connector portion 64, thereby maintaining the connection's reliability.

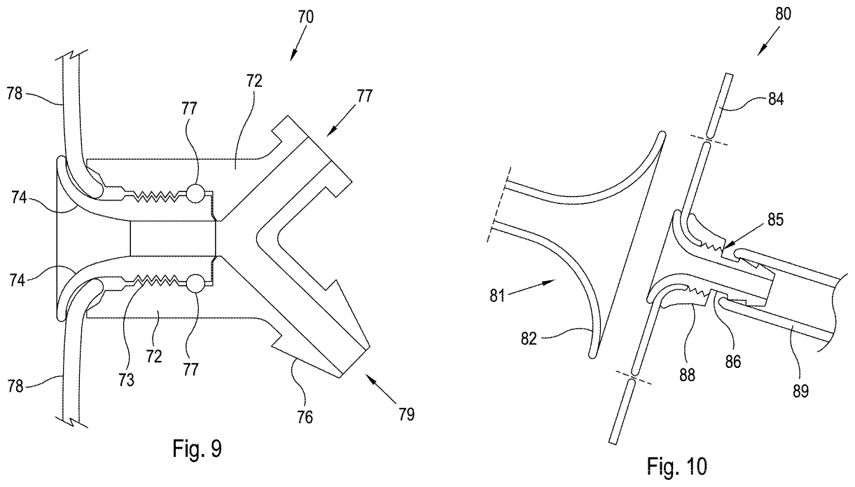

FIG. 9 shows a cross sectional view of a sixth variant of the connector of the present disclosure. The connector 70 comprises a connector body 72, a patch retainer 74 and a flexible patch 78.

The connector body 72 comprises a conduit connector 76 (comprising a barb) for connection to a conduit such as a perfusion tube, a de-airing port 77, and a female threaded inlet port 73. The inlet port 73 is in fluid communication with the perfusion port 79 of the barb 76 and the de-airing port 77.

The patch retainer 74 comprises a cylindrical portion that comprises a male threaded region, by which the patch retainer 74 can be screwed into the inlet port 73 of the first connector portion. At a proximal end of the male threaded region there is a groove, within which is received a o-ring seal member 77 (other types of seal member are also possible). The seal member 77 provides for sealing engagement between the first connector portion 72 and the second connector portion 74. The position of the seal member 77 is merely exemplary, and alternatives are possible in which the seal member 77 is disposed in contact with the flexible patch 78 and the connector body 72.

A distal portion of the patch retained portion 74 may be flared outward to form an annular recess between the second connector portion 74 and the first connector portion 72. The flexible patch 78 is secured in the annular recess between the first and second connector portion when the second connector portion 74 is screwed into the first connector portion 72. The perfusion patch 78 may comprise a PTFE material (or similar biocompatible material) and is configured to be joined to a vessel or organ by suturing. This may enable irregular flanges to be connected to which may be difficult to form a seal with using a clamping style of connector.

FIG. 10 shows a sixth variant of a connector 80 according to the present disclosure, comprising a connector body 86, patch retainer 88 and flexible patch 84 clamped between the connector body 86 and the patch retainer 88.

The connector body 86 comprises a conduit connector (comprising barb) at a first end for connection to a conduit (such as a ¼ inch tubing). The connector body 86 comprises a flared region at the second end (opposite to the first end). The connector body 86 comprises a male threaded portion 85 between the barb and the flared region. The patch retainer comprises a female threaded portion for engagement with the male threaded portion of the first connector portion 86. Rotation of the second connector portion 86 with threads 85 engaged moves the second connector portion 86 to clamp the perfusion patch 84 between the flared region of the first connector portion 86 and the second connector portion 88.

The perfusion patch 84 may comprise any suitable material, such as ePTFE.

An arterial patch 81 is shown in FIG. 10, adjacent to the flexible patch 84. The flexible patch 84 can be trimmed to remove excess (e.g. to match the size and shape of the flange 82 of the arterial patch 81). The flexible patch 84 and the flange 82 of the arterial patch 81 may be sealed together by suturing.

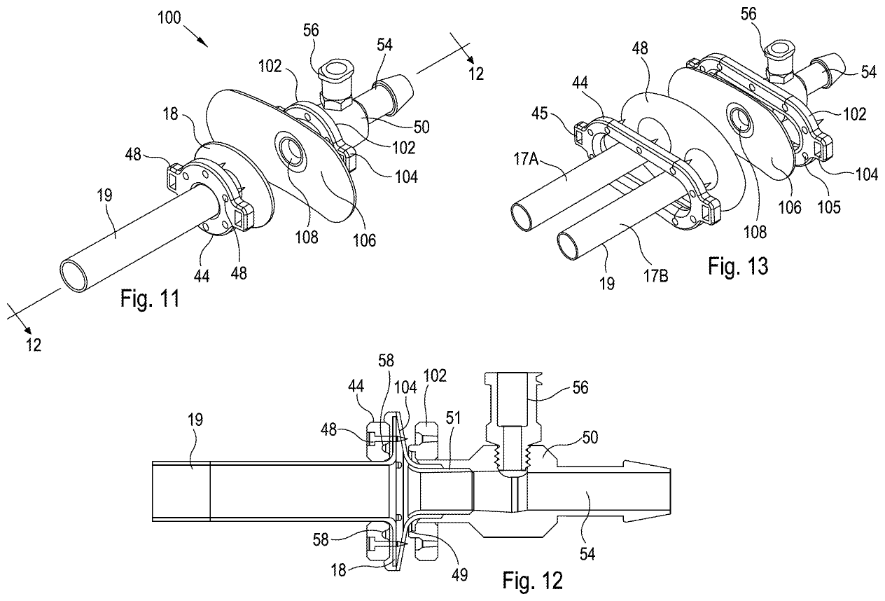

FIGS. 11 to 13 show a connector 100 according to an eighth variant of the present disclosure. The connector 100 comprises a first connector portion or connector body 50, second connector portion 44, and third connector portion 102. Similar to the connector of FIGS. 3 to 5, the connector portion 50 comprises a de-airing port 56 and a conduit connector 54 (comprising a barb). At the opposite end to the barb 54, the connector body 50 is connected to a flexible patch 106. The flexible patch 106 may be circular or generally oval shaped (an obroud shape is shown in FIGS. 11 and 13). The flexible patch 106 provides a large flat sealing region for sealing engagement with the flange 18 of the carrel patch 19. The flexible patch 106 is depicted as curved in FIGS. 11 to 13, but it will readily flex to conform with an underlying part under pressure. The flexible patch 106 may be formed from a different material to the connector body 50. The connector body 50 may be formed from a relatively hard polymeric material, for example with a Shore A hardness of at least 90. The flexible patch 106 may be formed from a soft polymeric material (e.g. ePTFE or similar) with a Shore A hardness of less than 60.

As shown in FIG. 12, the flexible patch 106 is retained between the connector body 50 and a patch retainer 51 and an inlet port of the connector body. The patch retainer 51 is in the shape of a tube section with a flared of flanged end. The patch retainer 51 is an interference fit with the inlet port of the connector body 50 and is configured to retain the flexible patch 106 in sealing engagement with the connector body 50, with the flexible patch 106 captured between the patch retainer 51 and the inlet port of the connector body 50.

The second connector portion 44 and third connector portion 102 are arranged on opposite sides of the flexible patch 106, with the carrel patch flange 18 between the second connector portion 44 and the flexible patch 106. The arrangements for securing the second connector portion 44 to the third connector portion 102 for the examples of FIGS. 11 and 13 are respectively as described with reference to FIGS. 3 and 4 except that the barbs pass through both the carrel patch flange 18 and the flexible patch 106 and are received in the third connector portion 102.

The connector shown in FIG. 13 is configured to connect a carrel patch 19 comprising a first vessel 17A and a second vessel 17B to a common barb 54 (e.g. for perfusion circulation via both vessels 19A, 19B).

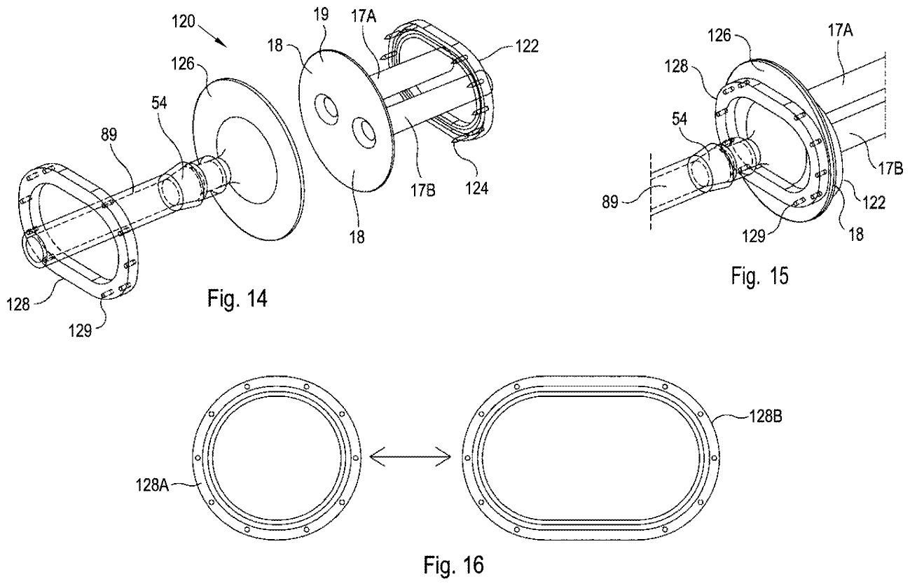

FIGS. 14-16 depict a connector 120 according to ninth variant of the present disclosure. The connector 120 is similar to the connector 60 depicted in FIG. 6, except that the connector 120 is configured to connect a carrel patch 19 having multiple blood vessels 17A, 17B to a barb 54 that can be coupled to a non-biological conduit 89 (e.g. flexible polymeric tubing).

In order to accommodate the blood vessels 17A, 17B the connector 120 is generally oblong in shape, but otherwise is configured similarly to the connector 60. The connector 120 comprises a first connector portion 126 that comprises a flat annular sealing region and which comprises a tapering region connecting the flat annular sealing region to the barb 54. The second and third connector portions 122, 128 are configured similarly to the second and third connector portions 64, 65 of the connector 60 of FIGS. 7 and 8, except that the second and third connector portions 122, 128 are obroud or generally oval in shape.

In some variants, the second and third connector portions 122, 128 may be deformable (or can be redesigned) from having a circular shape to having an oblong shape as depicted in FIG. 16.

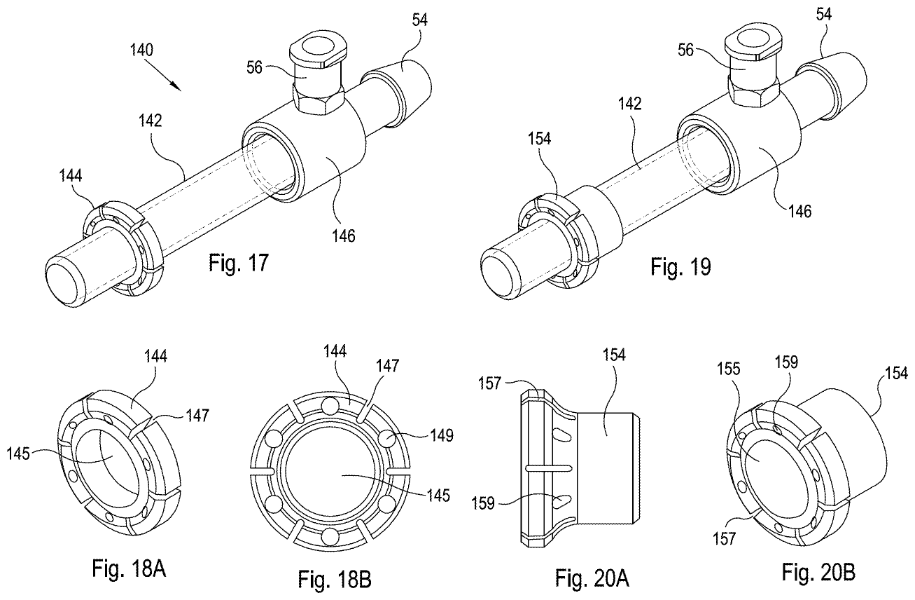

FIGS. 17, 18A, 18B, 19, 20A and 20B show a connector 140 according to a eleventh variant of the present disclosure. Connector 140 comprises spigot 142, connector body 146 and annular member 144, 154.

Connector body 146 comprises a conduit connector 54 (comprising barb) for connection to a conduit (such as tube for perfusion circulation) and a de-airing port 56. The conduit connector 54 is connected to and coaxial with a cylindrical body portion that is configured to receive the spigot 142 in sealing engagement therewith (e.g. by in interference fit or similar).

The annular member 144, 154 is fixed on the spigot 142 with an interference fit, so that the annular member 144, 154 can be repositioned axially along the spigot 142 with the application of sufficient force, but is held in place against forces that could be expected to arise as a result of pressure within the connector arising from perfusion (e.g. normothermic perfusion, which may comprise mean arterial pressures in the range of 70-75 mmHg).

The annular member 144, 154 comprises a plurality of radial slots 147, 157 spaced around the circumference of the annular member 144, 154 (e.g. equally spaced), with an axial through holes 149, 159 between each pair of adjacent radial slots 147, 157. The axial through holes 149, 159 and radial slots 147, 157 are capable of retaining sutures (e.g. when the sutures are threaded through the slots 147, 157 and/or holes 149, 159). Sutures may be used to secure the an anatomical conduit over the spigot 142 in sealing engagement therewith.

FIG. 21 shows a cannula 142 coupled to a connector 160. The connector 160 comprises a conduit connector (comprising barb) 164 having a perfusion port and a de-airing port 166. An insertion dilator 168 is inserted into the de-airing port. The de-airing port is coaxial with the lumen 142 where it couples to the connector 160 so that the insertion dilator 168 has a straight run through the connector 160 into the lumen 142. The insertion dilator 168 may be used to make it easier to slip a tip of an anatomical vessel over a portion of the connector 160 (e.g. a spigot or similar). In some embodiments the de-airing port may in the place of the conduit connector and the conduit connector may be in the place of the de-airing port.

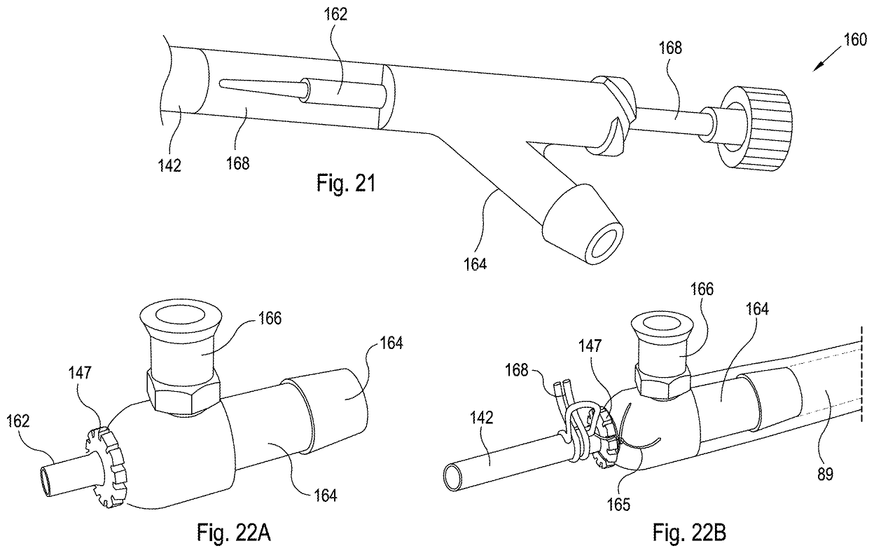

FIG. 22A shows a connector comprising a conduit connector (comprising barb) 164 at a first end for connection to a conduit (e.g. perfusion tubing) and a spigot 162 at a second opposite end of the connector. The connector comprises a de-airing port 166 between the first end and the second end. Immediately adjacent the cannula 162 is a flange comprising slots 147 for receiving a suturing thread.

FIG. 22B shows the connector of FIG. 22A with a conduit 89 coupled to the barb 164 and a lumen 142 (e.g. of a blood vessel) coupled in sealing engagement with the spigot 162. The lumen 142 is secured in engagement with connector by a spring clip 168 that urges the lumen 142 into sealing engagement with and external surface of the cannula 162 and by a suturing thread 165 that couples the lumen 142 to the radial slots of the flange adjacent to the cannula 142.

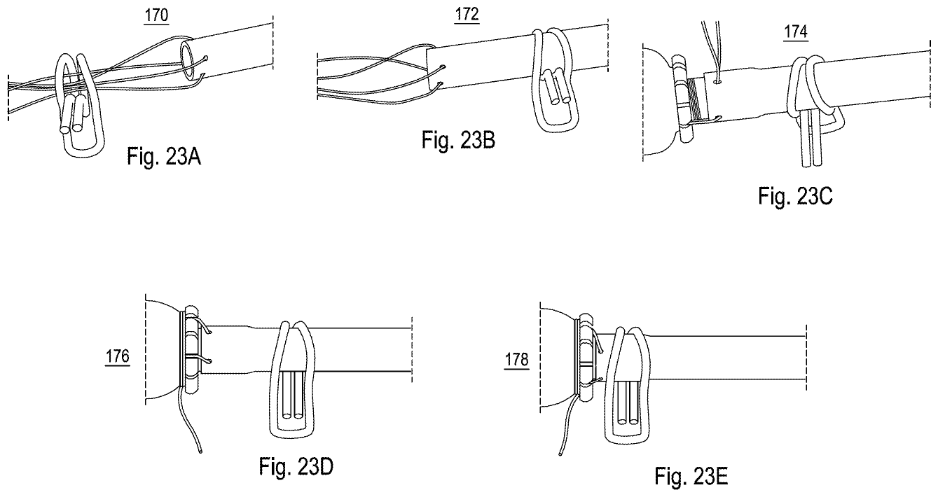

FIGS. 23A to 23E depict the process of applying the anatomical conduit (e.g. blood vessel) 142 to the cannula 162 of the connector shown in FIGS. 22A 22B. In FIGS. 23A and 23B 170, 172 a suturing thread is threaded through a tip of the anatomical conduit and a spring clip is placed on the lumen near the tip of the anatomical conduit. In FIG. 23C 174 the cannula 162 is inserted into the tip of the anatomical conduit, and in FIG. 23D 176 the suturing thread 165 is received in the radial slots 147. In FIG. 23E 178 the spring clip is moved onto the cannula 162 to retain the tip of the anatomical conduit in sealing engagement with the cannula 162.

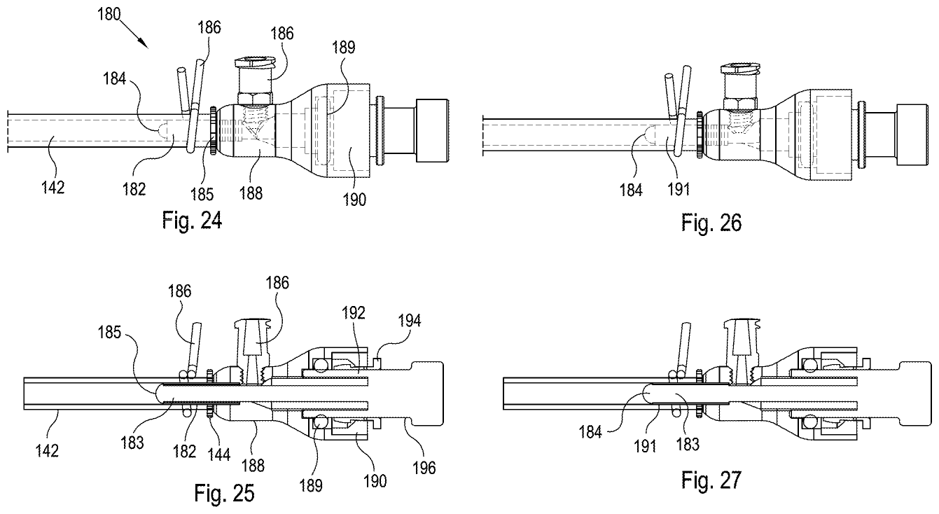

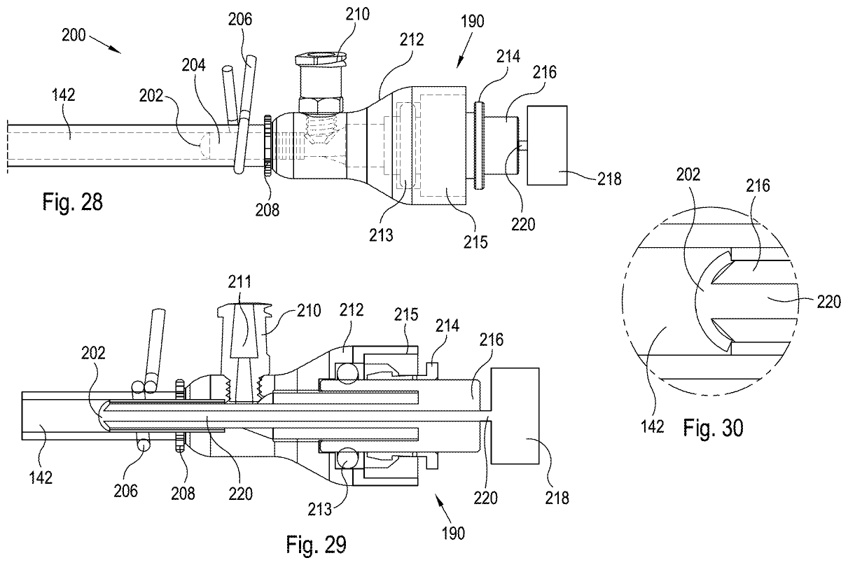

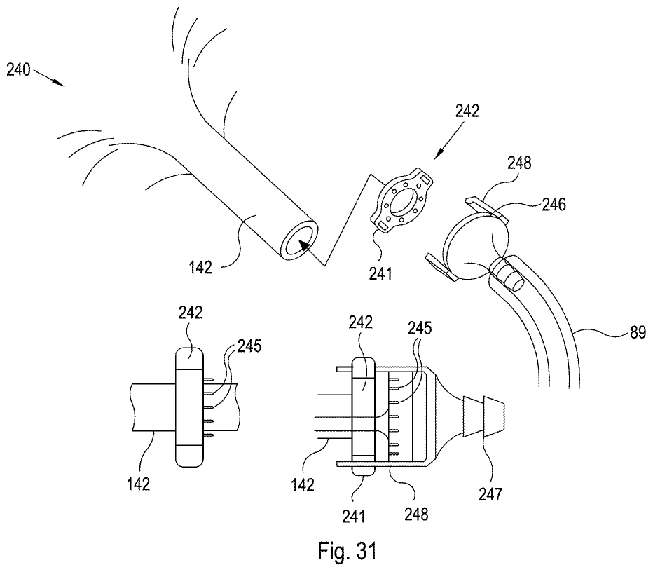

FIGS. 24 to 30 show a connector 180, 200. The connector 180, 200 is similar to that of FIGS. 22A and 22B except that the conduit connector comprises a quick connector instead of a barb. The connector 180, 200 comprises: de-airing port 186, 210 (e.g. comprising Luer lock 212), spigot 182, stylet 183, 220 slotted flange 185, 208, spring clip 185, 206, connector body 188, 212, quick connector 190 and stylet guide 196, 216.

The stylet guide 196, 214 is received in the quick connector 190 and retained by the quick connector collet 194, 214 in sealing engagement with the quick connector seal 189, 213. The stylet guide 196, 216 comprises an axial through hole receiving the stylet 183, 220. The stylet 183, 220 comprises an atraumatic stylet tip 184, 202 and can be inserted into the anatomical conduit 142 in order to open it and allow it to be more easily slipped over the spigot 184, 204. The anatomical conduit 142 may be retained in sealing connection with the spigot 184, 204 by sutures securing the anatomical conduit 142 to the slotted flange 185, 208, and/or using the spring clip 186, 206. After the anatomical conduit 142 is coupled to the connector 180, the stylet 183, 220 and stylet guide 196 may be removed from the quick connector 190 and a conduit (e.g. of a perfusion system) inserted into the quick connector 190 and thereby coupled to the connector 180, 200. The stylet 183, 220 may be moved axially using stylet handle 218.

The spigot in any of the preceding embodiments may comprise a solid wall (e.g. formed from stainless steel or other another suitable metal) or comprise a wire-wound cannula.

FIG. 31 illustrates a yet further variant, showing connector 240, comprising first connector portion 246 having projecting tabs 248 that removably interface with receiving tabs 241 of the second connector portion 242.

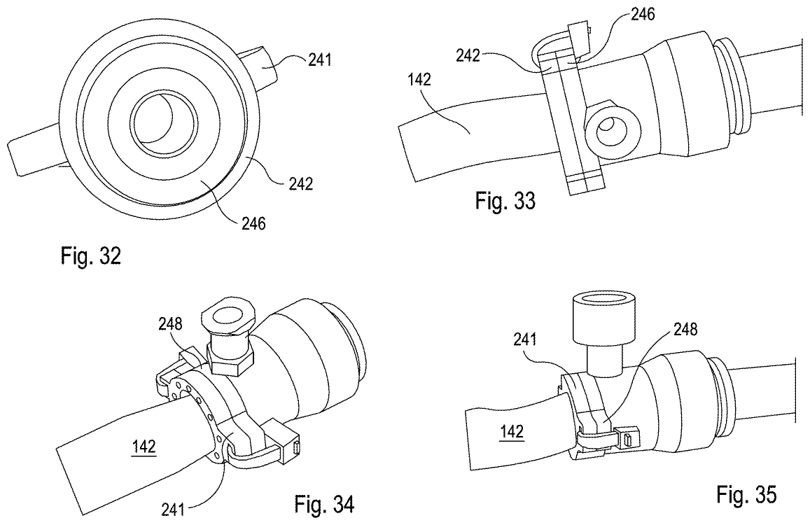

FIGS. 32 to 35 depict a further variant of a connector of the present disclosure which is similar to that of FIGS. 3 to 5, except that instead of the conduit connector comprising a barb, the conduit connector comprises a quick connector.

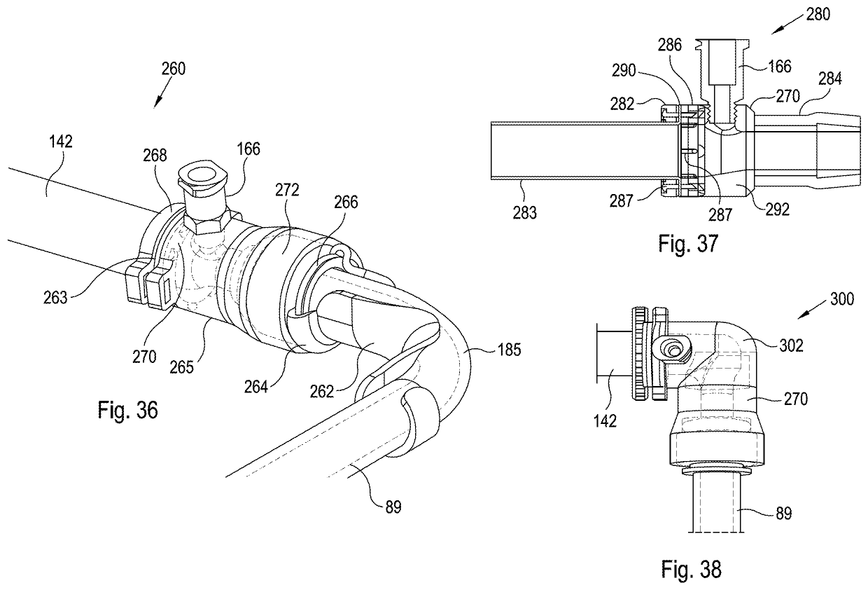

FIG. 36 depicts the connector of FIGS. 32 to 35 in combination with an conduit guide 262 which is configured to support the conduit 89 in a 90-degree bend with a relatively compact bend radius, which may be helpful in routing the conduit 89 in a perfusion context. Other types of conduit can also be used (e.g. different bend angles and so on).

The connector 260 comprises: de-airing port 166, projecting tabs 248, quick connector 272 (comprising collet 266), first connector portion 270 and second connector portion 268. The conduit guide 262 is clipped to the collet 266 of the quick connector 272.

FIG. 37 depicts a further variant showing connector 280, connecting anatomical conduit 283 (e.g. blood vessel comprising flange 290) to non-biological conduit 284. The connector 280 comprises: first connector portion 292, second connector portion 282, and barbed member 287. The connector 280 is similar to that shown in FIG. 3.

FIG. 38 depicts a further variant wherein the first connector portion 270 of connector 300 is an elbow connector, connecting an anatomical conduit 142 at 90 degrees to the non-biological conduit 89.

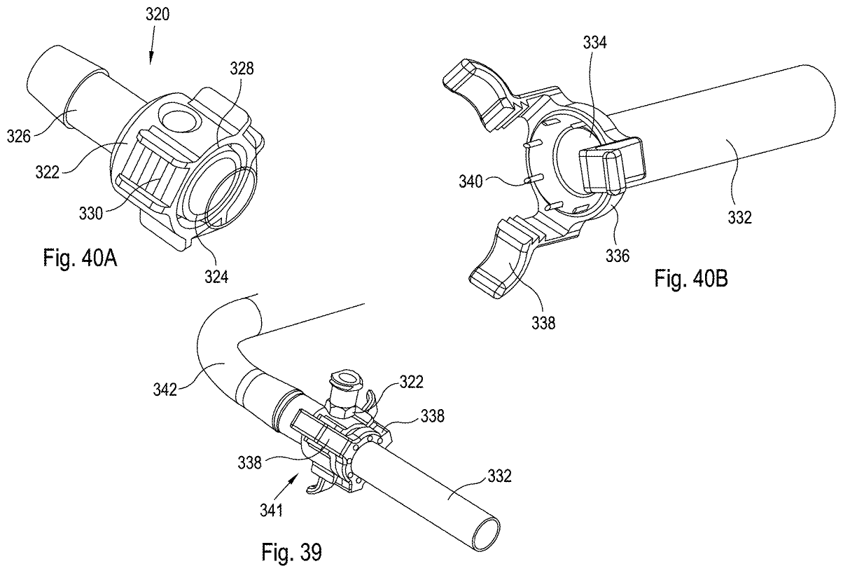

FIGS. 39, 40A and 40B depicts a further connector 320, which is similar to that of FIG. 37, except that the first connector portion 322 is clamped to the second connector portion 336 by three ratcheting fasteners 341 spaced apart on a perimeter of the connector 320. Each ratcheting fastener 341 comprises a pawl 338 formed unitarily with the second connector portion 336, the pawl 338 comprising a plurality of teeth. Each ratcheting fastener 341 further comprises fixed teeth 330 configured to engage with the teeth of the pawl to retain the first connector portion 322 clamped to the second connector portion 336, similar to the ratcheting fastener described with reference to FIGS. 1 and 2.

The anatomical conduit 332 comprises a flange 334 which is retained in position relative to the second connector portion 336 using barbed members 340. The connector comprises a conduit connector 326 comprising a barb and a de-airing port.

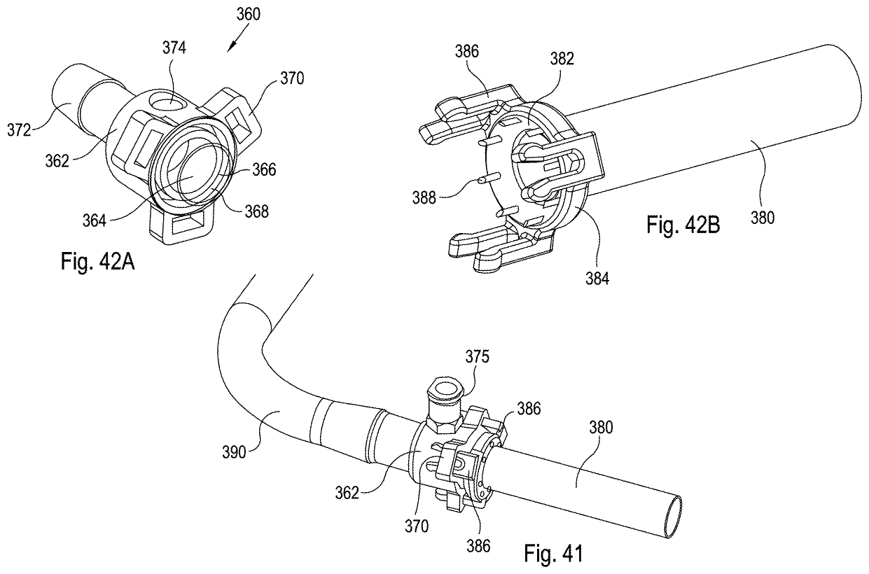

FIGS. 41, 42A and 42B depict a further connector 360, which is similar to that of FIG. 37, except that the first connector portion 322 is clamped to the second connector portion 336 by three catches 386 which are received in corresponding lugs 370. The catches 385 and lugs 370 are spaced apart on a perimeter of the connector 370. Each catch 385 comprises a pair of resiliently deformable members having a protrusion at a distal tip thereof, which engage with the lug 370 to secure the first connector portion 322 clamped to the second connector portion 384.

The anatomical conduit 380 comprises a flange 382 which is retained in position relative to the second connector portion 384 using barbed members 388. The connector 360 comprises a conduit connector 372 comprising a barb, and a de-airing port 374 comprising a Luer lock 375.

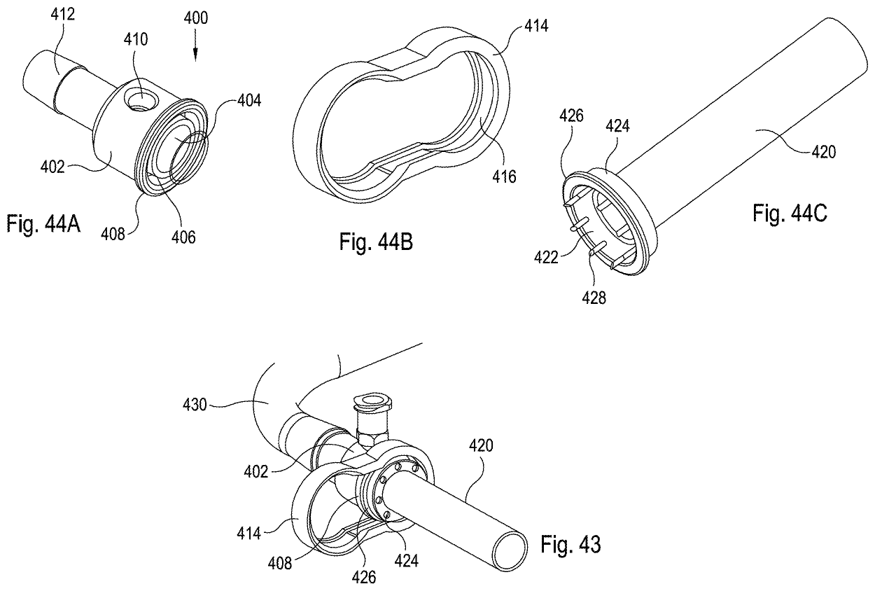

FIGS. 43, 44A and 44B depict a further connector 400, which is similar to that of FIG. 37, except that the first connector portion 402 is clamped to the second connector portion 424 by a sliding clamp 414 comprising a guide for laterally moving the second connector portion 424 into clamped engagement with the first connector portion 402.

The catches 385 and lugs 370 are spaced apart on a perimeter of the connector 370. Each catch 385 comprises a pair of resiliently deformable members having a protrusion at a distal tip thereof, which engage with the lug 370 to secure the first connector portion 322 clamped to the second connector portion 384.

The anatomical conduit 420 comprises a flange 422 which is retained in position relative to the second connector portion 424 using barbed members 428. The connector 400 comprises a conduit connector 412 comprising a barb, and a de-airing port 410 comprising a Luer lock.

The connectors disclosed herein may be configured for normothermic machine perfusion of an organ, capable of sealing against pressures of 75 mmHg or 10 kPa, and compatible with a perfusion fluid comprising red blood cells at body temperature.

In each of the connectors of the present disclosure the first connector portion, second connector portion and third connector portion (where present) may be separate components.

In each of the connectors of the present disclosure the connectors may be formed of a biocompatible material, such as PTFE, silicone or polyurethane (or at least portions of the connector that are in contact with the carrel patch or a fluid circulating through the connector may be made from a biocompatible material).

1. A connector for coupling an anatomical conduit to a non-biological conduit, the connector comprising:
a first connector portion comprising a conduit connector for coupling to a non-biological conduit;
a second connector portion; and
one or more ratcheting fasteners configured to clamp the second connector portion to the first connector portion with the portion of the anatomical conduit between the first connector portion and the second connector portion and in sealing engagement with a sealing region of the first connector portion.
